(19) United States
(12) Patent Application Publication
(10) Pub. No.: US 2024/0000612 A1
(43) Pub. Date: Jan. 4, 2024

(54) A Trephine Blade For Preparation Of Corneal Tissue For Endothelial Keratoplasty

(71) Applicant: Chameen Dilantha Samarawickrama, North Epping (AU)

(72) Inventor: Chameen Dilantha Samarawickrama, North Epping (AU)

(21) Appl. No.: 18/038,950

(22) PCT Filed: Nov. 26, 2021
(86) PCT No.: PCT/AU2021/051414

§ 371 Date: May 25, 2023

(30) Foreign Application Priority Data
Nov. 26, 2020 (AU) 2020904369
Nov. 18, 2021 (AU) 2021903727

(51) Int. Cl. A61F 9/013

(52) U.S. Cl. CPC A61F 9/0133; A61F 9/0136

(57) ABSTRACT

There is provided a trephine blade (10) for use in the preparation of a corneal graft for endothelial keratoplasty. The blade comprises a circular cutting edge (16) incorporating a notch formation (18) for cutting a correspondingly shaped asymmetric notch in the circumferential perimeter of donor corneal tissue to indicate correct orientation of the tissue in said endothelial keratoplasty. The notch formation can be defined by an inwardly directed cutting edge (20) of the blade and a further cutting edge (22) extending from an inner end of the inwardly directed cutting edge to the outer circumferential perimeter of the blade. In embodiments, the asymmetric notch in the circumferential perimeter of the donor corneal tissue is shaped to indicate whether the endothelial layer of the corneal tissue is facing upwardly or downwardly, when the corneal tissue is viewed from above. The endothelial keratoplasty in which the corneal graft may be used can be selected from DSAEK, DMEK, DLEK and DSEK.

### FIELD OF THE INVENTION

The present invention relates to a trephine blade for preparation of a donor corneal graft for use in eye surgery. Cutting devices comprising the blade are also provided together with methods for use of the blade.

### BACKGROUND TO THE INVENTION

Corneal transplantation is a key component in the treatment of corneal blindness and other corneal disease. The first iteration of corneal transplant involved the removal of the full thickness of the host cornea and replacement with a healthy full thickness donor cornea.

Several corneal surgical techniques have been developed in recent times resulting in significant advances in the treatment of diseases and conditions affecting the endothelium and associated tissues of the cornea, primary examples of which include Fuch's dystrophy, Posterior Polymorphous Membrane dystrophy and pseudophakic Bullous Keratopathy (PBK) amongst others. These techniques involve transplanting endothelial tissue of a donor cornea along with selected attached inner layer(s) of the cornea into the recipient's eye through a corneal or sclerocorneal incision following dissection of relevant tissue from the recipient eye, and are known as endothelial keratoplasty (EK)

Deep lamellar endothelial keratoplasty (DLEK) is an early form of EK which involves dissection of the posterior stromal lamella, Descemet's membrane and endothelium of the eye being treated and inserting a corneal graft comprising donor posterior stroma tissue, Descemet's membrane and endothelium. Later EK methods include Descemet's Stripping EK (DSEK) and Descemet's Membrane EK (DMEK). Descemet's Stripping Automated EK (DSAEK) is a variation of DSEK in which the donor corneal layers are separated using an automated microkeratome for provision of the graft tissue to improve the graft-host interface, resulting in faster visual recovery, and reduced incidence of patient astigmatism and graft failure.

However, each of these EK techniques require correct orientation of the corneal graft in the recipient's eye, namely with the graft endothelial layer facing toward the anterior chamber and iris. The graft tissue is generally inserted into the eye through the corneal or sclerocorneal incision in a folded condition. Once located in the anterior chamber, the graft tissue is unfolded by gentle manipulation. It is possible though for the graft to unfold in an incorrect orientation in which the endothelial side of the graft faces upwards (towards the host cornea) rather than downwards (towards the iris). Incorrect orientation of the graft leads to graft failure if not identified by the corneal surgeon.

As a means for identifying incorrect orientation of the graft in the eye, different numbers of circular cuts can be individually manually punched into each side of the circumferential perimeter of the graft. However, this results in an undesirable amount of corneal tissue being removed from the graft and an irregular circumference increasing the risk of potential dislocation of the graft from the recipient tissue and/or failure of the graft in those areas. In another method, a dye can be used to mark symbols (e.g., a letter in brail) to indicate graft orientation. The dye though may adversely affect tissue cells in the vicinity of the marked symbols and/or wash or fade from clear view. Each of these methods also involves additional steps in the handling of the graft for transplantation which is undesirable increasing the risk of damage to endothelial cells and other tissues of grafts.

### SUMMARY OF THE INVENTION

Broadly stated, in one aspect of the invention there is provided a trephine blade for use in the preparation of a corneal graft for endothelial keratoplasty, the blade comprising a circular cutting edge incorporating a notch formation for cutting a correspondingly shaped asymmetric notch in the circumferential perimeter of donor corneal tissue to indicate correct orientation of the corneal graft in said endothelial keratoplasty.

Typically, the asymmetric notch in the circumferential perimeter of the donor corneal tissue is shaped to indicate whether the endothelial layer of the corneal tissue is facing upwardly or downwardly when the corneal tissue is viewed from above.

Typically, the determination of the whether the endothelial layer of the corneal tissue is facing upwardly or downwardly is dependent on the orientation of the notch when viewed from above.

Typically, the notch formation is defined by an inwardly directed cutting edge of the blade and a further cutting edge extending from an inner end of the inwardly directed cutting edge to the outer circumferential perimeter of the blade.

Typically, the inwardly directed cutting edge and the further cutting edge are oriented at an acute angle relative to one another. Most usually, the acute angle is in a range of from about 70° to about 80° more preferably, is about 75°.

Typically, the inwardly directed cutting edge and the further cutting edge are both straight cutting edges.

Typically, the further cutting edge is longer in length than the inwardly directed cutting edge.

Typically, the inwardly directed cutting edge extends generally radially with respect to the longitudinal axis of the blade.

Typically, the notch formation of the blade is configured to form a notch in the circumferential perimeter of the donor corneal tissue that is generally in the shape of the number “7” or a stylised form thereof when the corneal tissue is viewed from above with the endothelium layer of the corneal tissue facing down, i.e., the correct orientation within the eye.

Typically, the blade is tubular with a peripheral side wall surrounding a through passageway extending from one end of the blade to an opposite end of the blade defining the cutting edge.

Typically, the notch formation is defined by a longitudinally directed channel formed in the peripheral side wall of the blade.

In particularly preferred embodiments, the channel extends from the one end of the blade to the opposite end of the blade.

Typically, the notch formation protrudes into the through passageway of the blade.

Typically, the outer diameter of the peripheral wall tapers in a direction from the one end of the blade to the cutting edge.

In other embodiments, the outer diameter of the blade can be generally constant along one end region of the blade to a middle region, and taper from the middle region to the opposite end of the blade defining the cutting edge. In still further embodiments, the outer diameter of the blade can be generally constant for the full length of the blade from the one end of the blade to the opposite end defining the cutting edge.

In at least some embodiments, the cutting edge of a trephine blade embodied by the invention can also include one or more further cutting formations for respectively cutting a correspondingly shaped formation in the circumferential perimeter of the donor corneal tissue. The one or more further cutting formations can be for independently forming an outwardly directed flap or a further notch in the periphery of the corneal tissue. In preferred embodiments, the one or more further formations in the perimeter of the corneal tissue can be located in proximity to the notch formed by the said notch formation of the blade or e.g., at a predetermined circumferential angle relative thereto, and so assist the surgeon in identifying that notch to confirm the orientation of the corneal graft following location of the graft in the recipient eye.

In another aspect there is provided a cutting device for the preparation of a corneal graft for endothelial keratoplasty, the device incorporating a trephine blade in accordance with the invention.

In another aspect of the invention there is provided a cutting device for the preparation of a corneal graft for endothelial keratoplasty, the device comprising a trephine blade having a circular cutting edge incorporating a notch formation for cutting a correspondingly shaped asymmetric notch in the circumferential perimeter of donor corneal tissue to indicate correct orientation of the corneal graft in said endothelial keratoplasty, the blade being retained by the device for the cutting of the donor corneal tissue by the blade.

Typically, the cutting device is a punch. In preferred embodiments the trephine blade is housed within the punch in use of the blade to cut the corneal tissue.

Typically, the blade is arranged for being driven toward the donor corneal tissue to effect the cutting of the donor tissue by the blade with operation of the punch.

In another aspect of the invention there is provided a method for preparing a corneal graft for use in endothelial keratoplasty, comprising the steps of:
providing a trephine blade for cutting donor corneal tissue to provide the corneal graft, the blade having a circular cutting edge incorporating a notch formation for cutting a correspondingly shaped asymmetric notch in the circumferential perimeter of the donor corneal tissue to indicate correct orientation of the corneal graft in said endothelial keratoplasty;
cutting the corneal tissue with the blade; and
retrieving the cut corneal tissue incorporating the notch.

The endothelial keratoplasty can, for example, be selected from the group consisting of DSAEK, DMEK, DLEK and DSEK corneal transplant methods.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers, integers or steps.

Any discussion of documents, acts, materials, devices, articles or the like that has been included in this specification is solely for the purpose of providing a context for the invention. It is not to be taken as an admission that any, or all, of these matters form part of the prior art base or were common general knowledge in the field relevant to the invention as it existed in Australia or elsewhere before the priority date of this application.

The features and advantages of the present invention will become further apparent from the following detailed description of exemplary embodiments of the invention together with the accompanying drawings.

### BRIEF DESCRIPTION OF THE ACCOMPANYING DRAWINGS

FIG. 1 is an isometric view of a trephine blade embodied by the invention;

FIG. 2 is a diagrammatic view illustrating the end profile of the cutting edge of the trephine blade of FIG. 1;

FIG. 3 is a plan view from above of donor corneal graft tissue cut to shape by trephine blade of FIG. 1 with the graft lying endothelial side face down;

FIG. 4 is an elevated diagrammatic view of the cutting block of a punch for cutting donor corneal tissue using a trephine blade embodied by the invention;

FIG. 5 is an elevated diagrammatic view of the punch top for mating with the cutting block of FIG. 4 in an upturned orientation and with the trephine blade mounted in position; and

FIG. 6 is an elevated side diagrammatic view of the punch top of FIG. 5.

### DETAILED DESCRIPTION OF EXEMPLARY EMBODIMENTS OF THE INVENTION

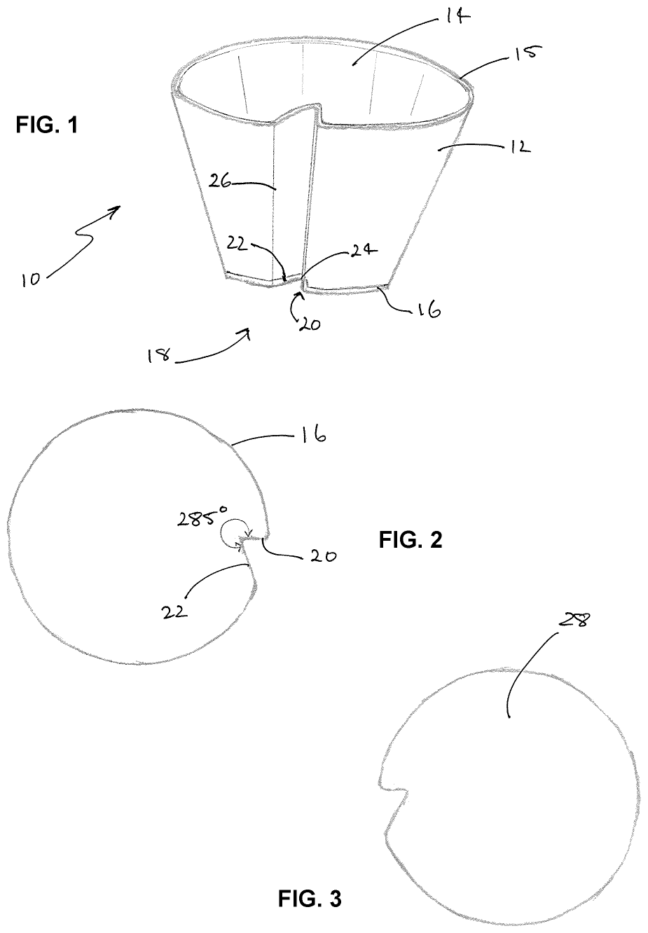

A trephine blade 10 embodied by the invention is illustrated in FIG. 1. The blade is tubular in form with a peripheral side wall 12 surrounding a through passageway 14 which extends from end 15 of the blade to an opposite end which defines a circumferential cutting edge 16. The cutting edge incorporates a notch formation 18 defined by an inwardly directed cutting edge section 20 and a further cutting edge section 22 extending from the inner end of the inwardly directed cutting edge to the outer circumferential perimeter of the blade. The inwardly directed cutting edge and the further cutting edge of the notch formation 18 meet at an apex 24 that protrudes into the through passageway of the blade.

As can also be seen from FIG. 1, the notch formation is defined by a longitudinally extending channel 26 formed in the peripheral side wall 12 that extends the full length of the blade from end 15 to its opposite end defining the cutting edge 16.

In the embodiment shown, the outer diameter of the peripheral side wall 12 tapers in a direction from the end 15 of the blade to the cutting edge 16. In other embodiments, the outer diameter of the blade can be generally constant from the end 15 of the blade to a middle region, and then taper from the middle region to the opposite end of the blade at which the cutting edge 16 is defined.

The inwardly directed cutting edge 20 and the further cutting edge 22 are both straight cutting edges in the embodiment shown though in other embodiments, one or both of those cutting edges may be independently slightly bowed. The bowing may for instance be either concave or convex in profile. Typically, the inwardly directed cutting edge 20 extends generally radially with respect to the longitudinal axis of the blade. To minimise corneal tissue loss, the inwardly directed cutting edge 20 and the further cutting edge 22 are oriented at an acute angle relative to one another. The acute angle is ideally in a range of from about 70° to about 80° and more usually, about 75°. The inclined further cutting edge 22 is therefore longer than the inwardly directed cutting edge and in preferred embodiments, the notch formation is configured to form an asymmetrical notch in the circumferential perimeter of the donor corneal tissue that is generally in the shape of the number “7” when the corneal tissue is viewed from above with the endothelium layer of the corneal tissue facing down.

The notch formation is sized for removal of less than about 3% and usually, about 2.5% or less, about 2% or less, or about 1.5% or less of the donor corneal tissue to be used as the corneal graft. That is, in the latter instance, the notch formation 18 is sized whereby 98.5% or more of the corneal tissue for use as the graft remains for transplantation into the recipient eye. More usually, the notch formation is sized to remove about 1.25% or less of the donor corneal tissue for use as the corneal graft, more usually about 1.0% or less, about 0.9% or less, about 0.85% or less, or 0.8% or less and most usually less than about 0.75% of the corneal tissue to be used as the graft.

The internal diameter 10 of the trephine blade at the cutting edge end of the blade not accounting for the notch can be of any dimension of conventional tubular trephine blades such as e.g., 6.0 mm, 6.25 mm, 6.5 mm, 6.75 mm, 7.00 mm, 7.25 mm, 7.5 mm, 7.75 mm, 8.0 mm, 8.25 mm, 8.5 mm, 8.75 mm or 9.0 mm or more. A range of trephine blades in accordance with the invention of different sizes may therefore be provided in which the internal diameter of the blades at their cutting edge may increase in 0.25 mm or 0.5 mm increments as indicated above. The thickness of the peripheral side wall 12 of the blade 10 may be that of conventionally known tubular trephine blades, and the blade 10 may be fabricated from surgical grade stainless steel or other suitable metal or metal alloy.

A diagrammatic end view illustrating the profile of the cutting edge 16 of the trephine blade 10 of FIG. 1 is shown in FIG. 2, whilst corneal tissue 28 cut to size by the trephine blade 10 with the endothelial side of the corneal tissue facing down is shown in FIG. 3. In this orientation the notch cut into the corneal tissue will therefore have the profile of the number “7” when viewed from above the cornea with the endothelium facing downwards. Thus, when the final corneal graft tissue is inserted and properly orientated and located in the anterior chamber of the eye during endothelial keratoplasty (EK), the number “7” shaped notch can be discerned by the surgeon when looking into the recipient eye from the front of the eye. If a number “7” cannot be identified it indicates to the surgeon that the graft is incorrectly orientated and the graft has been flipped over so that the endothelium side of the graft is wrongly facing towards the interior of the cornea.

In the corneal tissue shown in FIG. 3, the length of the section of the tissue cut by the inwardly directed cutting edge 20 of the blade 10 is about 0.5 mm whilst the length of the section of the tissue cut by the further cutting edge 22 of the blade is about 1.5 mm, the reflex angle between those edges of the tissue being about 285°.

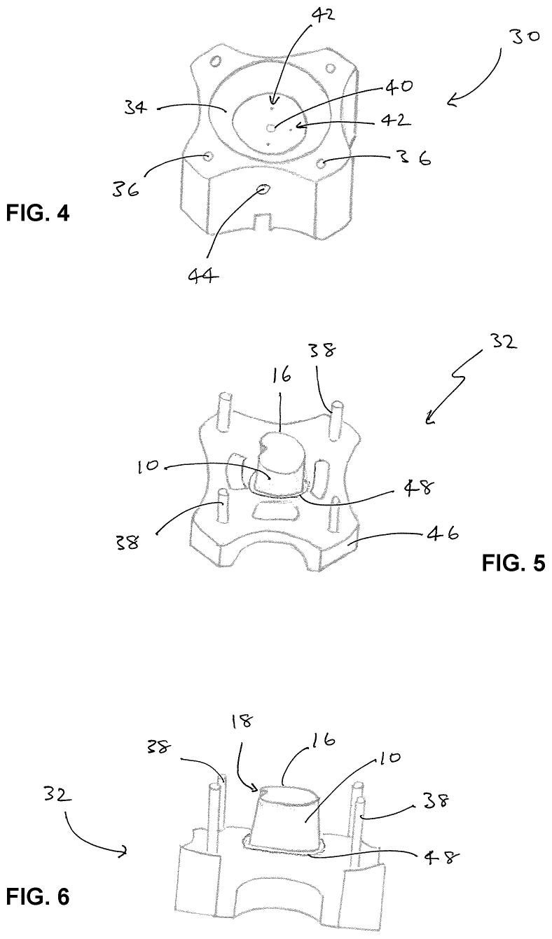

The cutting block 30 and a blade assembly 32 respectively forming the base and plunger of a punch for cutting donor corneal tissue with a blade embodied by the invention is illustrated in FIGS. 4 to 6.

The cutting block 30 comprises a comprises a centrally located concave well 34 and openings 36 spaced apart from one another around the block for respectively receiving a corresponding post 38 of the blade assembly. A through passageway 40 opening to a vacuum chamber (not shown) of the cutting block that is located under the well 34 is located centrally in the base of the well. Further through passageways 42 which also open to the vacuum chamber are equidistantly spaced apart from one another around the central passageway 40. In use, a spring-loaded syringe is connected to the cutting block via medical grade silicone inserted into opening 44 formed in a side of the cutting block for creating suction in the vacuum chamber as further described below.

The blade assembly 32 includes a plunger cap 46 in which is defined a centrally located circular groove 48 in which the end 15 of the trephine blade 10 is seated with the cutting edge 16 of the blade exposed. The blade is retained in the groove in a press fit and as also shown, the metal posts 38 are spaced around the periphery of the cap to align with the openings 36 of the cutting block. In at least some embodiments, the plunger cap includes a through passageway from the top of the cap to the underside face of the cap so as to open into the interior through passageway 14 of the blade 10 to permit viewing of the corneal tissue from above by the surgeon or technician. The cutting block 30 and the blade assembly 32 may be fabricated from a stiff plastics material such as high-density polyethylene (HDPE) or high-density polypropylene though any suitable plastics material can be utilised for the intended purpose.

In preparation for the cutting of the corneal tissue in the punch, the required tissue layers of a donor corneoscleral button for the proposed EK corneal surgery (e.g., DMEK) are generally partially peeled as a single thickness of tissue from the remaining corneal tissue to greater than about 50% to about 75% of the distance across the cornea to form a corneal flap in the known manner. The corneal flap is then laid back over the exposed corneal tissue layer and the cornea is placed generally centrally within the well of the cutting block with the epithelial side of the cornea in contact with the cutting block (i.e., endothelial side up).

Prior to the placement of the corneal button in the cutting block, the spring-loaded syringe is connected to the vacuum chamber of the cutting block via the silicone tubing and the plunger of the syringe is pushed all the way into its barrel and held in that position. After placing the corneal button into the well of the cutting block as above, the button is gently pressed down to centre and seat the button against the floor of the well. Once the corneal button is appropriately seated in the well, the plunger of the syringe is released to create a vacuum in the vacuum chamber of the cutting block and thereby hold the corneal button against the well floor.

To cut the corneal tissue with the trephine blade 10, the blade assembly is gently lowered onto the cutting block with reception of the posts 38 in the corresponding openings 36 until the blade 10 rests on the endothelial face on the cornea. The cap of the blade assembly is then gently pressed downwardly to cut the corneal tissue with the blade before withdrawing the cap and removing the blade assembly from the cutting block. At this point, the vacuum in the vacuum chamber of the cutting block can be released, the scleral rim trimmed from donor corneal tissue removed with the use of forceps, and the cut-to-size donor cornea removed from the well. Cutting devices of the above type which may be used or adapted for use with a trephine blade embodied by the invention include Barron vacuum donor cornea punches™ (Barron Precision Instruments Inc., Grand Blanc, Michigan, USA).

The cutting of the donor corneal tissue as above can be undertaken either at an eye or tissue bank by a suitably trained technician or e.g., by the surgeon on site at the surgery. As will be understood, once the corneal tissue has been cut to size with a trephine blade embodied by the invention, the peeling of the corneal tissue can be completed to obtain the final corneal graft for transplantation to the patient.

As described above, the asymmetric notch cut into corneal graft ideally forms the number “7” when the graft is viewed from above with the endothelial side of the graft facing downwardly. Once inserted into the eye of the recipient, the lenticule can be orientated with ease using known techniques.

Thus, when transplanted, the correct orientation of the corneal graft is endothelial side down so as to face the anterior chamber of the eye. In this orientation the notch in the graft forms the number “7” when viewed from the front of the eye, irrespective of the rotational position of the graft. Hence, if the surgeon can discern a number “7” shaped notch in the perimeter of the corneal graft (again irrespective of the rotational orientation of the graft) following insertion of the graft into the eye as described above, the graft has been orientated correctly in the eye. If, however, the number “7” shaped notch cannot be discerned by the surgeon (and instead shows a reverse number “7”), the surgeon will know the graft has been located incorrectly, and needs to be flipped within the eye to the correct “endothelial side down” orientation. The identification of the orientation of the notch can be assisted by staining of the corneal tissue or graft with a suitable dye, e.g., trypan blue (0.05% v/v) or Membrane Blue Dual™ (e.g., Trypan blue (0.15% v/v), Brilliant blue (0.025% v/v) and polyethylene glycol (4% v/v)), which normally occurs prior to cutting the donor corneal tissue to size as described above.

In at least some embodiments of a trephine blade as described herein, the cutting edge 16 of the blade may include one or more further cutting formations for respectively cutting a correspondingly shaped formation in the circumferential perimeter of the donor corneal tissue, in addition to the notch formation 18. Each additional cutting formation can be for independently forming an outwardly directed flap or a further notch in the circumferential perimeter of the donor corneal tissue. The flap or further notch can be symmetrical or asymmetric and may be of any shape deemed appropriate. The flap or further notch may, for example, be arcuate, semi-circular, convex, concave, crescent shaped, U-shaped, or generally V-shaped as applicable amongst other possible shapes. In the instance the further cutting formation is for cutting an outwardly directed flap in the circumferential perimeter of the donor corneal tissue it is preferable that the flap be curved or arcuate in form.

A further cutting formation of the blade in at least some embodiments may be located in proximity to the notch formation 18 or at a predetermined circumferential angle either clockwise or anti-clockwise relative to notch formation 18, e.g., at an angle of 25°, 30°, 35°, 40°, or 45° or more. In preferred such embodiments, a further cutting formation of the blade may be located at about a 90° or 180° angle to the notch formation 18. Knowing the relative position of the flap or notch in the corneal graft cut by the further cutting formation relative to the asymmetric notch formed by cutting formation 18 can assist the eye surgeon or ophthalmologist to identify that asymmetric notch during the corneal transplantation operation, at follow up after surgery and during subsequent routine checks on the status of the graft. The flap or notch cut by the further cutting formation of the trephine blade can therefore act as a locational formation in the circumferential perimeter of the corneal graft. This can be of particular assistance should failure or rejection occur in peripheral region(s) of the graft.

In the event the further cutting formation is for forming a further notch in the donor corneal tissue the cutting formation will desirably be dimensioned to minimise the amount of tissue removed. Generally, the amount of the tissue removed by the further cutting edge will be similar to the amount removed by the notch formation 18. When the further cutting formation is for forming an outwardly directed flap in the circumferential periphery of the donor corneal tissue, the surface area of the flap may be greater than that removed by the notch formation 18. The flap may for instance extend a distance of up to 2 mm or 3 mm or more at its furthest radial distance from the notional circumference of the corneal tissue when cut to size by the blade. The term “flap” as used in herein is to be construed in the sense of a protruberance of tissue from the notional circumferential perimeter of the donor corneal tissue cut by the trephine blade.

A further cutting formation of the blade may be defined by an inwardly directed channel or outwardly directed rib in the peripheral side wall 12 of the trephine blade. The channel or rib may extend the full length or only part length of the blade.

In still other embodiments, the notch formation 18 and/or a further cutting formation may be defined by inwardly or outwardly directed pouching as applicable in the end region defining the cutting edge of the blade.

In at least some embodiments of a cutting device as described herein the circular groove 48 may be configured or profiled for reception of the end 15 of the blade to be seated within the groove in a specific rotational orientation of the blade. For example, the groove may be correspondingly profiled to mate with notch formation 18 of the blade and/or further cutting formation(s) if provided, to assist retention of the blade and inhibit rotation of the blade within the groove.

As a further option, the blade assembly of an embodiment of the invention may include a protrusion for reception in the notch formation or a further cutting formation of the blade in order to restrict the orientation of the blade and/or rotational movement of the blade about the longitudinal axis of the blade.

The larger outer diameter of the end 15 of the trephine blade 10 as illustrated in FIG. 1 facilitates handling of the blade.

In still further embodiments, the opposite end to the cutting edge of a trephine blade in accordance with the invention may be closed-ended forming a cap of the blade for being pressed to drive the blade to cut the donor corneal tissue in use of the blade. The closed “cap” end region may form a head portion of the blade to facilitate the use of the blade. In at least some forms, the peripheral circumference of the head of the blade may be knurled, grooved or e.g., ribbed for easier handling of the blade.

Whilst a particular type of punch for the cutting of the donor corneal tissue has been described above, a trephine blade embodied by the invention may also be used with other types of punches in which the blade is retained and housed in the punch in use, and which is driven to cut the donor tissue such as by operation of a plunger or otherwise to size and shape the tissue to obtain a corneal graft incorporating a asymmetric notch in its circumferential perimeter as described herein. For example, in some embodiments of cutting devices contemplated herein, the end 15 of the blade may be held in a holder and a hand-operated wheel or ring rotated to extend or drive the blade to cut the donor corneal tissue, and all such arrangements are expressly provided for. In still other embodiments, the trephine blade may be seated or secured in a holder wherein the holder is manually aligned with the donor corneal tissue and driven down whilst being held to cut the donor tissue to size, though such punch arrangements are preferably used in combination with a guide system for guidance of the holder/blade. Such guide systems may for instance comprise a frame structure that mounts to a base on which the donor corneal tissue is placed in use and wherein the frame structure has a centrally located opening disposed for being located over the base to receive the blade whereby the blade is aligned with the corneal tissue. As with the cutting block illustrated in FIG. 4, the corneal tissue can be positioned in a curved well defined in the upper face of the base for the cutting of the tissue by the blade.

From the above description, it will be appreciated that embodiments of a trephine blade in accordance with the invention may provide for one or more of the following advantages:
The asymmetric notch can be cut into the donor corneal tissue/corneal graft in a single cut at the same time the tissue is cut to size for transplantation in the recipient eye;
Cutting and sizing the donor corneal tissue in a single step saves time in preparing the corneal graft and reduces the risk of user error;
The use of a trephine blade embodied by the invention may also reduce central endothelial loss due to reduced tissue handling;
The asymmetric notch in the corneal graft provides for rapid identification of the correct orientation of the graft and thereby also correct placement of the graft in the recipient eye, and so acts to aid the correct orientation of the graft in the eye reducing user error in orientation of the graft in the eye;
A corneal graft incorporating an asymmetric notch in accordance with the present disclosure can be used in connection with e.g., DSAEK, DMEK, DLEK and DSEK corneal transplant methods;
Once inserted, the corneal graft behaves as a standard circular graft and can be manipulated without the need for further training of the corneal surgeon; and
Once attached, the asymmetric notch can be identified in the graft in the future so the surgeon or attending ophthalmologist can be confident the corneal graft was inserted in the recipient eye in the correct orientation and in the event the graft were to not recover and/or ultimately fail, can determine whether this was due to incorrect orientation of the graft or as a result of primary graft failure.

Although a number of embodiments of the invention have been described above it will be understood that various modifications and changes may be made thereto without departing from the invention. The above-described embodiments are therefore only illustrative and are not to be taken as being restrictive.

1. A trephine blade for use in the preparation of a corneal graft for endothelial keratoplasty, the blade comprising a circular cutting edge incorporating a notch formation for cutting a correspondingly shaped asymmetric notch in the circumferential perimeter of donor corneal tissue to indicate correct orientation of the corneal graft in said endothelial keratoplasty.

2. The blade of claim 1, wherein the asymmetric notch in the circumferential perimeter of the donor corneal tissue is shaped to indicate whether the endothelial layer of the corneal tissue is facing upwardly or downwardly, when the corneal tissue is viewed from above.

3. The blade of claim 1, wherein the notch formation is defined by an inwardly directed cutting edge of the blade and a further cutting edge extending from an inner end of the inwardly directed cutting edge to the outer circumferential perimeter of the blade.

4. The blade of claim 3, wherein the inwardly directed cutting edge and the further cutting edge are oriented at an acute angle relative to one another.

5. The blade of claim 4, wherein the acute angle is in a range of from about 70° to about 80°.

6. (canceled)

7. The blade of claim 3, wherein the inwardly directed cutting edge and the further cutting edge of the notch formation are both straight cutting edges.

8. The blade of claim 3, wherein the inwardly directed cutting edge extends generally radially with respect to the longitudinal axis of the blade.

9. The blade of claim 3, wherein the notch formation of the blade is configured to form a notch in the circumferential perimeter of the donor corneal tissue that is generally in the shape of the number “7” when the corneal tissue is viewed from above with the endothelium layer of the corneal tissue facing down.

10. The blade of claim 1, wherein the cutting edge of the blade further includes at least one further cutting formation for cutting a correspondingly shaped formation in the circumferential perimeter of the donor corneal tissue.

11. (canceled)

12. The blade of claim 10, wherein the further cutting formation is located in proximity to the notch formation or at a predetermined circumferential angle relative thereto.

13. The blade of claim 1, wherein the notch formation is configured for removing about 1.5% or less of the corneal donor tissue.

14. (canceled)

15. The blade of claim 1 being tubular with a through passageway extending from one end of the blade to the opposite end of the blade defining the cutting edge of the blade.

16. The blade of claim 15, wherein the notch formation protrudes into the through passageway of the blade.

17. The blade of claim 1, wherein the notch formation is defined by a longitudinally directed channel formed in the peripheral side wall of the blade.

18. The blade of claim 17, wherein the channel extends from the one end of the blade to the opposite end of the blade.

19. A cutting device for the preparation of a corneal graft for endothelial keratoplasty, the device comprising a trephine blade having a circular cutting edge incorporating a notch formation for cutting a correspondingly shaped asymmetric notch in the circumferential perimeter of donor corneal tissue to indicate correct orientation of the corneal graft in said endothelial keratoplasty, the blade being retained by the device for the cutting of the donor corneal tissue by the blade.

20-21. (canceled)

22. The device of claim 19 being a punch.

23. The device of claim 22, wherein the blade is housed within the punch in use.

24. The device of claim 19, wherein the blade is arranged for being driven toward the donor corneal tissue to effect the cutting of the donor tissue for preparation of the corneal graft with operation of the device.

25. A method for preparing a corneal graft for use in endothelial keratoplasty, comprising the steps of:
providing a trephine blade for cutting donor corneal tissue to provide the corneal graft, the blade having a circular cutting edge incorporating a notch formation for cutting a correspondingly shaped asymmetric notch in the circumferential perimeter of the donor corneal tissue to indicate correct orientation of the corneal graft in said endothelial keratoplasty;
cutting the corneal tissue with the blade; and
retrieving the cut corneal tissue incorporating the notch.

26-32. (canceled)
